(19) United States
(12) Patent Application Publication
(10) Pub. No.: US 2026/0188500 A1
(43) Pub. Date: Jul. 2, 2026

(54) Hardware-Anchored Federated Consensus System for Edge-Sealed Gradient Aggregation and Distributed Predetermined Change Control Plan Execution

(71) Applicant: George William Bickerstaff, III, Greenwich, CT (US)

(72) Inventor: George William Bickerstaff, III, Greenwich, CT (US)

(21) Appl. No.: 19/546,364

(22) Filed: Feb. 22, 2026

(51) Int. Cl. G16H 50/20; G07C 13/00; H04L 9/32

(52) U.S. Cl. CPC G16H 50/20; G07C 13/00; H04L 9/3247

(57) ABSTRACT

A decentralized, hardware-anchored consensus network for securely training and regulating medical artificial intelligence across multiple institutions. The system utilizes a provenance-linked aggregation engine that strictly accepts learning data only if it carries an unforgeable cryptographic seal generated directly at the medical sensor edge, blocking synthetic deepfakes. The aggregated global model is evaluated homomorphically against FDA Predetermined Change Control Plan boundaries stored in a global manifest lock. A distributed quorum of hospital hardware nodes must cryptographically vote to confirm the updated model's safety before deployment. Once a supermajority is reached, a multi-node safety synchronization deploys the update globally. A hardware-enforced global rollback circuit constantly monitors the network, capable of autonomously bypassing local operating systems to revert all hospitals to a previous model version if real-world performance breaches safety thresholds, ensuring absolute, verified regulatory compliance across the federated grid.

### FIELD

The present invention relates to hardware-secured telecommunications networks for collaborative medical artificial intelligence. More particularly, the invention provides a decentralized cryptographic consensus architecture that aggregates machine-learning data exclusively from cryptographically edge-sealed medical sensors, and utilizes a distributed hardware quorum to verify global model updates against FDA Predetermined Change Control Plans before network-wide deployment.

### BACKGROUND

While federated learning allows hospitals to train AI collaboratively, current software-based networks cannot mathematically guarantee the physical origin of the training data or ensure that the combined global model remains within authorized regulatory safety bounds. If one hospital submits data from a degraded scanner, or if the aggregated global model violates a safety threshold, the entire network is corrupted. There is an unmet need for a distributed hardware architecture that acts as an automated regulatory body, demanding point-of-capture data sealing and multi-institutional cryptographic consensus before allowing an AI model to evolve.

### PRACTICAL APPLICATION & NON-OBVIOUSNESS

The claimed invention integrates distributed cryptographic consensus into a hardware-secured network architecture that materially alters how model gradients are processed and validated. By forcing a hardware-level interlock between edge-sealed data provenance and distributed Predetermined Change Control Plan (PCCP) evaluation, this system achieves autonomous, zero-trust regulatory compliance governed by human-configured safety parameters across multiple institutions, as demonstrated in Example 1. This significantly mitigates the catastrophic risks of unverified global model drift, providing a quantifiable technological improvement over generic software aggregation that satisfies 35 U.S.C. § 101. Furthermore, this hardware integration addresses long-felt, unmet needs for cross-institutional regulatory synchronization since the inception of the FDA PCCP framework, providing strong secondary indicia of non-obviousness that explicitly bolsters the Graham factors under 35 U.S.C. § 103.

### DEFINITIONS

Cross-Institutional Equity Matrix: A hardware-accelerated module that mathematically balances learning contributions across different hospital demographics. It prevents large urban hospitals from overwhelming the data submitted by smaller rural clinics. It ensures the resulting global artificial intelligence remains medically accurate for all patient populations.

Decentralized PCCP Validator: A distributed logic circuit spanning multiple physical hospitals that evaluates proposed AI updates. It requires a mathematical supermajority to agree that a global model update remains within authorized safety limits. It operates entirely independently of any single hospital's local administration.

Distributed PCCP Quorum: The formalized collective of authorized hardware nodes participating in the global network. This group acts as a decentralized regulatory body governed by strict cryptographic voting rules. It must achieve mathematical consensus before any new algorithmic baseline is legally adopted.

Edge-Sealed Gradient: An artificial intelligence learning pattern that carries a permanent cryptographic signature originating directly from a physical medical scanner. This signature proves the data was never altered between the moment of capture and the moment of network ingestion. It completely eliminates the ingestion of synthetic or intercepted medical data.

Global Manifest Cryptographic Lock: A hardware-enforced vault located on the central aggregation server that stores the master regulatory boundaries. It dictates the absolute maximum allowable deviation for any global model update. It physically prevents the distribution of any algorithmic update that exceeds these hardcoded limits.

Hardware-Enforced Global Rollback: An emergency network protocol triggered when a newly deployed global model fails distributed real-world validation. It instantly commands all connected hospitals to revert to the previous cryptographically signed model version. It bypasses local hospital operating systems to ensure immediate network-wide safety synchronization.

Homomorphic Boundary Evaluator: A secure processor that checks encrypted AI model updates against safety thresholds without decrypting the underlying data. It allows the network to verify the safety of a proposed medical model while maintaining absolute patient privacy. It ensures intellectual property and patient records remain completely hidden during regulatory checks.

Multi-Node Safety Synchronization: The automated process of aligning the active artificial intelligence version across every participating hospital simultaneously. It utilizes zero-latency cryptographic handshakes to swap the old model for the new model at the exact same millisecond. It guarantees that no two hospitals are ever utilizing different versions of the master algorithm.

Provenance-Linked Aggregation Engine: A central combining server that refuses to merge data unless every single input contains a valid hardware sensor seal. It physically drops incoming learning patterns that cannot mathematically prove their point of origin. It ensures the global master model is built exclusively on pristine, verified clinical evidence.

Synthetic Demographic Padding: A secure mathematical technique used to protect the privacy of highly unique patient populations during federated learning. It injects mathematically valid but synthetic statistical noise to obscure rare genetic or phenotypic traits. It ensures that advanced artificial intelligence can learn from rare diseases without exposing the identities of individual patients.

### BRIEF DESCRIPTION OF THE DRAWINGS

Referring now to the drawings submitted separately in compliance with 37 CFR 1.84, the following figures illustrate the preferred embodiments of the invention.

### DETAILED DESCRIPTION OF THE DRAWINGS

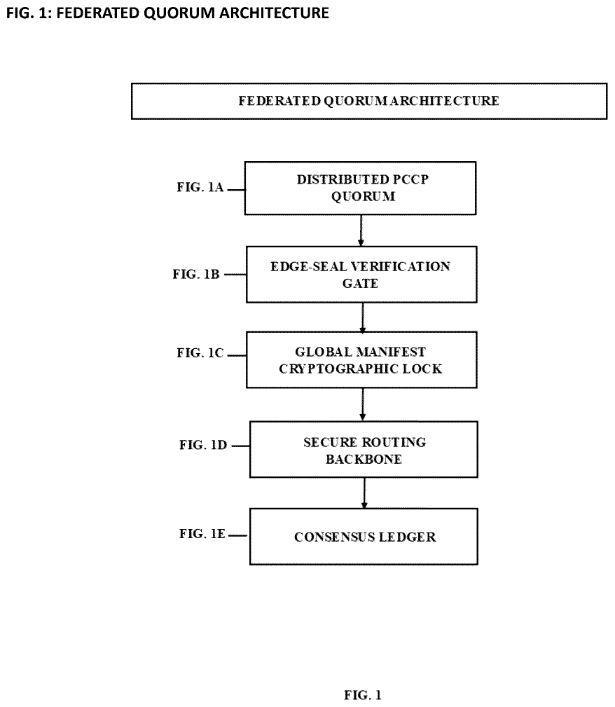

### FIG. 1: Federated Quorum Architecture

Referring now to FIG. 1A, the Distributed PCCP Quorum acts as the network's governing body. It requires cryptographic agreement from multiple hospitals before approving any global software update. This prevents any single compromised institution from forcing a dangerous global model change.

Referring now to FIG. 1B, the Edge-Seal Verification Gate inspects every incoming learning pattern. It demands a cryptographic signature originating directly from the physical medical scanner. It immediately rejects gradients generated from unverified or synthetic data sources.

Referring now to FIG. 1C, the global manifest cryptographic lock holds the ultimate regulatory boundaries for the entire network. It mathematically defines the maximum safe limits for the artificial intelligence's evolution. It physically blocks the transmission of any model that violates these federal guidelines.

Referring now to FIG. 1D, the Secure Routing Backbone manages the flow of encrypted data between participating institutions. It completely isolates the highly sensitive medical network from standard internet traffic. It utilizes dynamic pathway shifting to prevent external actors from intercepting the learning patterns.

Referring now to FIG. 1E, the Consensus Ledger permanently records the voting outcome of every distributed quorum decision. It provides a flawless, unalterable history of exactly how and why a global model was updated. It serves as the ultimate legal proof of regulatory compliance for federal auditors.

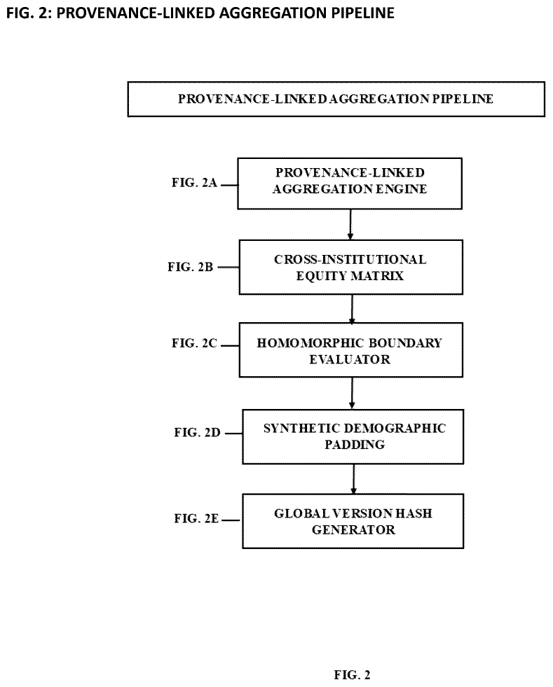

### FIG. 2: Provenance-Linked Aggregation Pipeline

Referring now to FIG. 2A, the Provenance-Linked Aggregation Engine serves as the highly secure digital mixing chamber. It carefully merges the verified learning patterns from hundreds of different hospitals. It will only initiate the combination process if every single input passes the strict edge-seal verification.

Referring now to FIG. 2B, the Cross-Institutional Equity Matrix analyzes the demographic breakdown of the incoming data. It actively weights the learning patterns to prevent any specific demographic from dominating the global model. It mathematically ensures the resulting medical tool is highly accurate for all human populations.

Referring now to FIG. 2C, the Homomorphic Boundary Evaluator tests the newly combined model while it remains heavily encrypted. It executes complex safety checks without ever exposing the raw mathematical weights to network administrators. It completely secures the intellectual property of the collaborative research consortium.

Referring now to FIG. 2D, the Synthetic Demographic Padding module activates when highly rare medical conditions are processed. It mathematically obscures the learning data to protect the identities of patients with highly unique genetic profiles. It ensures compliance with the strictest international digital privacy laws.

Referring now to FIG. 2E, the Global Version Hash Generator finalizes the newly approved master artificial intelligence model. It applies a permanent cryptographic seal derived from the combined signatures of the voting quorum. It creates an unforgeable digital stamp that participating hospitals use to verify the update.

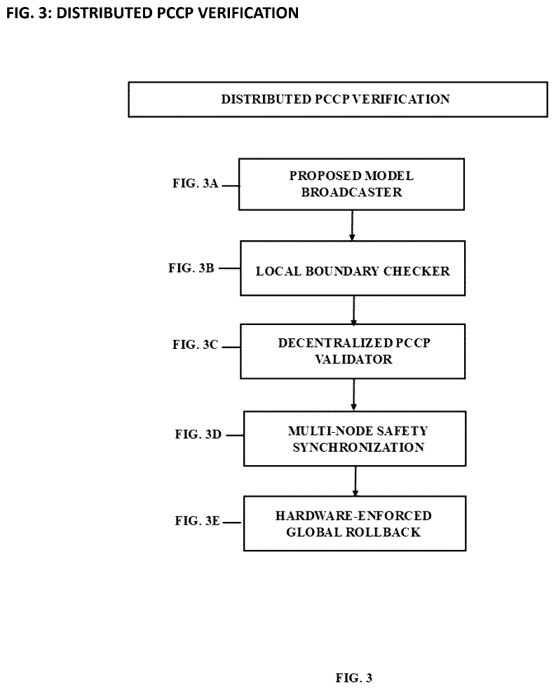

### FIG. 3: Distributed PCCP Verification

Referring now to FIG. 3A, the Proposed Model Broadcaster sends the newly combined, unapproved algorithm to the quorum nodes for testing. It distributes the software inside secure, temporary hardware enclaves located at each participating hospital. It prevents the proposed software from interacting with live patients during the voting phase.

Referring now to FIG. 3B, the Local Boundary Checker executes the proposed global model against the hospital's local safety rules. It verifies that the globally proposed changes do not violate any regional medical standards. It generates a cryptographic pass or fail token based entirely on the hardware's mathematical analysis.

Referring now to FIG. 3C, the Decentralized PCCP Validator tallies the cryptographic voting tokens from across the entire network. It mandates that a pre-programmed supermajority of hospitals must return a passing token. It instantly terminates the update process if the supermajority consensus is not mathematically achieved.

Referring now to FIG. 3D, the Multi-Node Safety Synchronization protocol initiates the live deployment of the approved model. It coordinates a simultaneous software swap across the entire global hospital network. It guarantees absolute version parity so no hospital is left running outdated diagnostic tools.

Referring now to FIG. 3E, the Hardware-Enforced Global Rollback remains on standby immediately following a network-wide update. It constantly monitors for severe clinical anomalies reported by the local hospital nodes. It instantly reverts the entire global network to the prior model version if a safety threshold is breached.

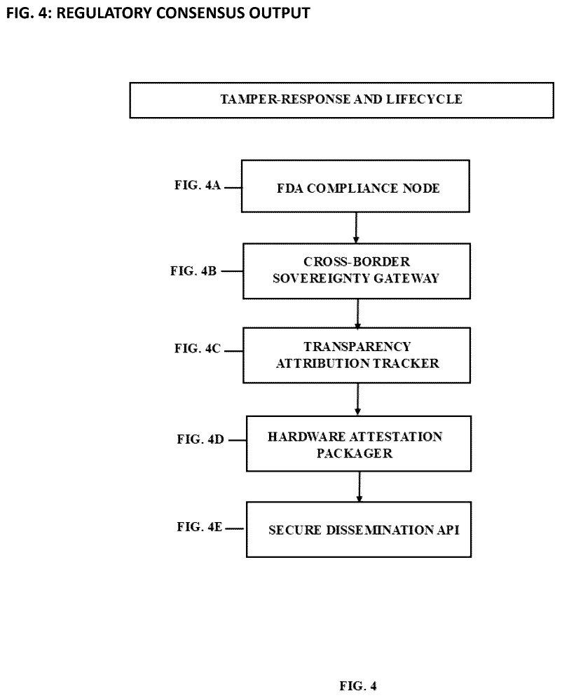

### FIG. 4: Regulatory Consensus Output

Referring now to FIG. 4A, the FDA Compliance Node automatically formats the distributed quorum's voting data for federal review. It translates the highly complex cryptographic math into plain-language continuous learning reports. It dramatically reduces the administrative burden placed on the collaborative hospital network.

Referring now to FIG. 4B, the Cross-Border Sovereignty Gateway manages international legal differences between collaborating hospitals. It mathematically translates European privacy mandates so they seamlessly integrate with American data standards. It allows global medical collaboration without violating local digital jurisdictions.

Referring now to FIG. 4C, the Transparency Attribution Tracker specifically maps which hospitals contributed to which algorithmic improvements. It provides clear, mathematical proof of how a specific clinical capability was developed. It allows the consortium to properly compensate institutions that provide high-value medical data.

Referring now to FIG. 4D, the Hardware Attestation Packager collects physical processor proofs from every node that voted on an update. It permanently binds these hardware signatures to the finalized regulatory report. It proves to auditors that the global update was authorized by physical machines, not software bots.

Referring now to FIG. 4E, the Secure Dissemination API provides a controlled access point for authorized government regulators. It allows federal inspectors to instantly download the cryptographically sealed compliance reports. It completely modernizes how federal agencies monitor continuous learning medical devices.

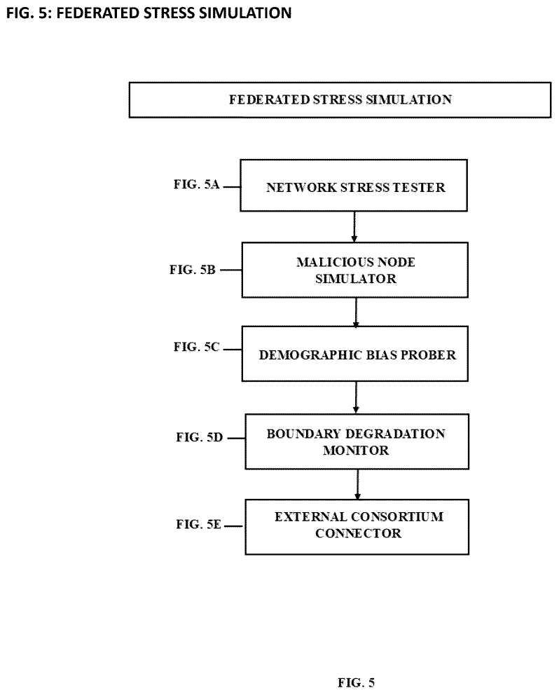

### FIG. 5: Federated Stress Simulation

Referring now to FIG. 5A, the Network Stress Tester artificially simulates massive internet outages across the federated hospital grid. It verifies that the distributed quorum can still safely process algorithmic votes during natural disasters. It mathematically proves the extreme resilience of the decentralized medical network.

Referring now to FIG. 5B, the Malicious Node Simulator intentionally attempts to inject falsified, non-sealed data into the aggregation engine. It physically confirms that the Edge-Seal Verification Gate operates at zero-latency under active cyberattack. It acts as an automated, continuous penetration testing protocol for the system.

Referring now to FIG. 5C, the Demographic Bias Prober attempts to skew the global model by feeding it heavily biased learning patterns. It ensures the Cross-Institutional Equity Matrix successfully mathematically balances the artificial intelligence. It guarantees the final medical algorithm will not discriminate against minority populations.

Referring now to FIG. 5D, the Boundary Degradation Monitor uses predictive math to estimate when the global model will naturally become outdated. It actively alerts the participating hospitals to prepare for an upcoming continuous learning cycle. It ensures the network remains proactive rather than reactive regarding software maintenance.

Referring now to FIG. 5E, the External Consortium Connector allows brand new hospital systems to safely join the established network. It initiates a rigorous hardware interrogation before issuing the new hospital a valid voting token. It securely expands the size and power of the collaborative research grid.

### VI. EXAMPLES OF ENABLEMENT

Example 1: Cross-Border PCCP Consensus for a Global Oncology Model. A consortium of twenty hospitals across the US and Europe collaborates to update a lung cancer AI model. Each hospital captures new patient data, sealing it immediately at the MRI scanner (Edge-Sealed Gradients). When the hospitals submit their homomorphically encrypted learning patterns, the Provenance-Linked Aggregation Engine verifies the hardware seal of every gradient, physically dropping any data lacking a true MRI-scanner origin. The combined model is then passed to the Distributed PCCP Quorum. The Homomorphic Boundary Evaluator checks the encrypted global model against the Global Manifest Cryptographic Lock, ensuring the model's new staging accuracy does not deviate beyond FDA-approved tolerance limits. Because a supermajority of the 20 hardware nodes cryptographically attest that the updated model is safe, the Multi-Node Safety Synchronization instantly deploys the update globally, seamlessly satisfying both FDA and GDPR requirements.

Example 2: Hardware-Enforced Global Rollback of a Triage Algorithm. An international network updates its ER triage AI following a successful Distributed PCCP Quorum vote. However, due to an unforeseen regional variation in how a specific heart medication is administered in a newly joined territory, the updated model begins demonstrating a sharp drop in accuracy at three specific hospitals. These three hospitals generate severe anomaly reports that are instantly broadcast to the network. The central network detects that this collective failure breaches the safety parameters defined in the Global Manifest Cryptographic Lock. The Hardware-Enforced Global Rollback is immediately triggered, bypassing all local hospital software administration to force a simultaneous, network-wide reversion to the previous cryptographically signed model version within one second, completely neutralizing the clinical risk.

What is claimed:

1. A hardware-anchored federated consensus system for distributed medical artificial intelligence, comprising: a provenance-linked aggregation engine configured to accept localized gradient updates exclusively when said updates contain an edge-sealed cryptographic signature generated directly by a physical medical sensor; a global manifest cryptographic lock storing the maximum safety deviation limits for the network; a distributed predetermined change control plan quorum comprising a plurality of authorized hardware nodes; and a decentralized validator configured to deploy a global artificial intelligence model update exclusively upon verifying that the aggregated model mathematics strictly comply with the global manifest cryptographic lock via a cryptographic supermajority vote from the hardware nodes.

2. A method for securely aggregating and regulating continuous learning artificial intelligence across multiple healthcare institutions, comprising the steps of: receiving homomorphically encrypted gradient updates exclusively from verified medical edge sensors; utilizing a cross-institutional equity matrix to mathematically balance learning contributions across diverse patient demographics; evaluating the combined global artificial intelligence model against hardware-stored predetermined change control plan boundaries using a homomorphic boundary evaluator; obtaining a cryptographically signed safety consensus from a distributed hardware quorum; and executing a multi-node safety synchronization to deploy the globally updated model simultaneously across all participating network nodes.

3. A decentralized safety assurance and emergency rollback architecture for federated clinical networks, comprising: an interconnected network of physical trusted execution environments; a central consensus ledger that permanently records cryptographic voting attestations from said execution environments; and a hardware-enforced global rollback circuit hardwired to continuously monitor aggregate clinical anomaly reports across the network, wherein the rollback circuit is configured to autonomously bypass local node operating systems to force an instantaneous, network-wide reversion to a prior cryptographic version of an artificial intelligence model if predetermined safety parameters are breached.

4. The system of claim 1, wherein the provenance-linked aggregation engine instantly and irreversibly deletes any incoming learning pattern that lacks a mathematically valid edge-sealed cryptographic signature, completely neutralizing the ingestion of synthetic deepfake data.

5. The system of claim 1, wherein the distributed predetermined change control plan quorum executes its cryptographic voting protocols strictly within hardware-isolated secure enclaves to prevent local software-level interference.

6. The system of claim 1, further comprising a cross-border sovereignty gateway configured to translate divergent international data privacy mandates into unified hardware logic prior to global model combination.

7. The method of claim 2, further comprising the step of injecting synthetic demographic padding into the learning patterns to mathematically obscure the identities of patients possessing highly unique or rare phenotypic traits.

8. The method of claim 2, wherein the multi-node safety synchronization utilizes hardware-synchronized atomic clocks to execute the model transition across all global nodes within exactly one millisecond.

9. The method of claim 2, wherein the cryptographically signed safety consensus requires a multi-signature validation from at least seventy-five percent of the active distributed hardware quorum.

10. The architecture of claim 3, wherein the consensus ledger is formatted to automatically generate interoperable, mathematically proven continuous learning reports compliant with FDA Predetermined Change Control Plan documentation standards.

11. The architecture of claim 3, wherein the hardware-enforced global rollback executes the complete network-wide model reversion in under one second to prevent mass clinical misdiagnosis.

12. The method of claim 2, wherein the enforcement of edge-sealed gradients and distributed quorum validation directly resolves long-felt clinical regulatory needs by mathematically guaranteeing the physical origin and collaborative safety of decentralized artificial intelligence training.
